(19) United States
(12) Patent Application Publication
(10) Pub. No.: US 2023/0293183 A1
(43) Pub. Date: Sep. 21, 2023

(54) FLOW DIVERTER DELIVERY SYSTEMS AND ASSOCIATED METHODS

(71) Applicant: Neromedica Inc., Mt. Pleasant, SC (US)

(72) Inventors: Narlin Beaty, Mt. Pleasant, SC (US); Mario Heintze, Mt. Pleasant, NC (US); Thomas Friedrich, Mt. Pleasant, NC (US)

(21) Appl. No.: 18/185,943

(22) Filed: Mar. 17, 2023

Related U.S. Application Data
(63) Continuation-in-part of application No. PCT/US2023/014400, filed on Mar. 2, 2023.
Application No. PCT/US2023/014400 is a continuation-in-part of application No. PCT/US2023/014853, filed on Mar. 8, 2023.
(60) Provisional application No. 63/321,069, filed on Mar. 17, 2022.
(60) Provisional application No. 63/315,904, filed on Mar. 2, 2022.
(60) Provisional application No. 63/317,937, filed on Mar. 8, 2022.

(51) Int. Cl. A61B 17/12; A61F 2/86

(52) U.S. Cl. CPC A61B 17/12118; A61B 17/12172; A61F 2/86; A61B 2017/12054

(57) ABSTRACT

A flow diverter delivery system having a catheter, a pusher wire slidably disposed within the catheter, a distal guide linearly coupled to a distal end of the pusher wire, and a flow diverter anchor coupled to the distal guide. A flow diverter having an undeployed and a deployed configuration is mechanically coupled to the flow diverter anchor and, when in the deployed configuration, includes a low-porosity distal cap, a transverse flow section coupled to the distal cap, and a linear support body coupled to the transverse flow section. The low-porosity distal cap is structurally configured to divert at least a portion of blood through the transverse flow section. When the flow diverter is exposed from the catheter, radial movement of the flow diverter away from the flow diverter anchor disengages the mechanical coupling between the flow diverter and the flow diverter anchor.

### CROSS-REFERENCE TO RELATED APPLICATIONS

This application claims the benefit of U.S. Provisional Patent Application No. 63/321,069, filed on Mar. 17, 2022, which is incorporated herein by reference in its entirety. This application is also a continuation-in-part of International Patent Cooperation Treaty Application No. PCT/US2023/014400, filed Mar. 2, 2023, which claims the benefit of U.S. Provisional Application Ser. No. 63/315,904, filed Mar. 2, 2022, and is also a continuation-in-part of International Patent Cooperation Treaty Application No. PCT/US2023/014853, filed Mar. 8, 2023, which claims the benefit of U.S. Provisional Application Ser. No. 63/317,937, filed Mar. 8, 2022, each of which is incorporated herein by reference in its entirety.

### BACKGROUND

Various medical devices are commonly implanted into humans for many medical conditions, which often involve physiological structures that are in need of intervention. Numerous implantable devices have been developed for treating such conditions, such as guidewires, catheters, medical device delivery systems (e.g., for stents, grafts, replacement valves, occlusive devices, etc.), and the like. For an aneurism, for example, a portion of a wall of a blood vessel can grow or otherwise form an outward recess. When such a recess is located, such that the blood flows into the recess under some pressure, the recess can continue to grow outwardly. Such outward growth can cause pressure on surrounding tissue, impede the functionality of the physiological structure where the recess has formed, and eventually rupture, thus causing a potential health risk or even death in the affected subject. Several of the aforementioned devices are commonly used to treat such conditions.

### BRIEF DESCRIPTION OF THE DRAWINGS

FIG. 1 illustrates a view of an aneurism at a blood vessel bifurcation;

FIG. 2 illustrates a view of an implant being delivered to a blood vessel in accordance with an example embodiment;

FIG. 3 illustrates a view of a flow diverter delivery system in accordance with an example embodiment;

FIG. 4A illustrates a view of a flow diverter in accordance with an example embodiment;

FIG. 4B illustrates a distal region of a flow diverter in accordance with an example embodiment;

FIG. 4C illustrates an inside view of a portion of a flow diverter in accordance with an example embodiment;

FIG. 5 illustrates an isometric view of a flow diverter in accordance with an example embodiment;

FIG. 6A illustrates a proximal looking view of a distal region of a flow diverter in accordance with an example embodiment;

FIG. 6B illustrates a side view of a distal region of a flow diverter in accordance with an example embodiment;

FIG. 6C illustrates a proximal looking view of a distal region of a flow diverter in accordance with an example embodiment;

FIG. 6D illustrates a side view of a distal region of a flow diverter in accordance with an example embodiment;

FIG. 7 illustrates a view of a flow diverter in accordance with an example embodiment;

FIG. 8 illustrates a proximal looking view of a distal region of a flow diverter in accordance with an example embodiment;

FIG. 9A illustrates a view of a flow diversion system having a flow diverter releasably coupled to a delivery device in accordance with an example embodiment;

FIG. 9B illustrates a view of a flow diversion system having a flow diverter shown nearing release from a delivery device in accordance with an example embodiment;

FIG. 10A illustrates a view of a flow diverter releasably coupled to a delivery device that is being positioned at an aneurism of a blood vessel bifurcation in accordance with an example embodiment;

FIG. 10B illustrates a view of a flow diverter having been released and deployed at an aneurism of a blood vessel bifurcation in accordance with an example embodiment.

FIG. 11A illustrates an isometric view of a flow diverter delivery system in accordance with an example embodiment.

FIG. 11B illustrates an isometric view of a flow diverter delivery system in accordance with an example embodiment.

FIG. 12A illustrates a view of a braided wire proximal transverse opening in accordance with an example embodiment;

FIG. 12B illustrates a view of braided wire making a mechanical connection with an implant anchor in accordance with an example embodiment;

FIG. 12C illustrates a view of the proximal end of an implant loaded into an implant delivery device in accordance with an example embodiment;

FIG. 13A illustrates a view of an implant mechanically secured in an implant delivery device in accordance with an example embodiment;

FIG. 13B illustrates a view of an implant nearing release from an implant anchor device in accordance with an example embodiment;

FIG. 13C illustrates a view of an implant released from an implant anchor device in accordance with an example embodiment; and

FIG. 14 illustrates a view of an implant delivery device in accordance with an example embodiment.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only and is not intended to be limiting. Unless defined otherwise, all technical and scientific terms used herein have the same meaning as commonly understood by one of ordinary skill in the art to which this disclosure belongs. Also, the same reference numerals appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, patterns, material examples, etc., to provide a thorough understanding of various embodiments. One skilled in the relevant art will recognize, however, that such detailed embodiments do not limit the overall concepts articulated herein but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 1%, and in some cases less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments described herein are, for example, capable of operation in other orientations than those illustrated or otherwise described herein.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art.

As used herein, the term “wire” can refer to a single wire or a bundle of wires, unless the context clearly indicates otherwise. As such, a structure described as a “braided wire” can refer to a braided single wire or a braided bundle of wires.

As used herein, “porosity” is defined as the fraction of the surface area of voids (pores) over the total surface area. In other words, Pt (%)=(Total Surface Area−Solid Surface Area)/(Total Surface Area)×100%.

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

Various medical conditions involve physiological structures that are in need of intervention, treatment, or repair. In many such situations, a portion of a wall of a vessel, duct, tissue, or the like, can grow or otherwise form a recess from the lumen side of the structure outward, or in other words, bulge outward from the structure. When such a recess is located where a biological fluid flows into the recess under some pressure, the recess can continue to grow outwardly. Such outward growth can cause pressure on surrounding tissue, impede the functionality of physiological structures adjacent to where the recess has formed, and eventually rupture, thus causing a potential health risk or even death in the affected subject. Specific nonlimiting examples of physiological structures can include pulmonary, cerebral, thoracic, and peripheral vasculature, as well any affected tube, duct, tissue, or the like, including hepatic, digestive, and renal systems.

As one specific example, a cerebral aneurysm is a weak or thin spot on an artery in the brain that bulges out and fills with blood. Aneurysms represent a significant health risk, including neurological effects from the resulting pressure on surrounding tissue as well as from rupture. A ruptured aneurysm can lead to hemorrhagic stroke, brain damage, coma, and even death. The size, location, and type of the aneurysm can be a significant factor in the severity of the health risk to the affected patient.

Cerebral aneurysms, particularly those that are very small, do not bleed or cause other health problems initially, but often have the potential to do so if steps are not taken to curtail the bulging and weakening of blood vessel walls. These types of aneurysms are often detected during imaging tests for suspected neural problems or other medical conditions. Cerebral aneurysms can occur anywhere in the brain, but many form in the major arteries along the base of the skull.

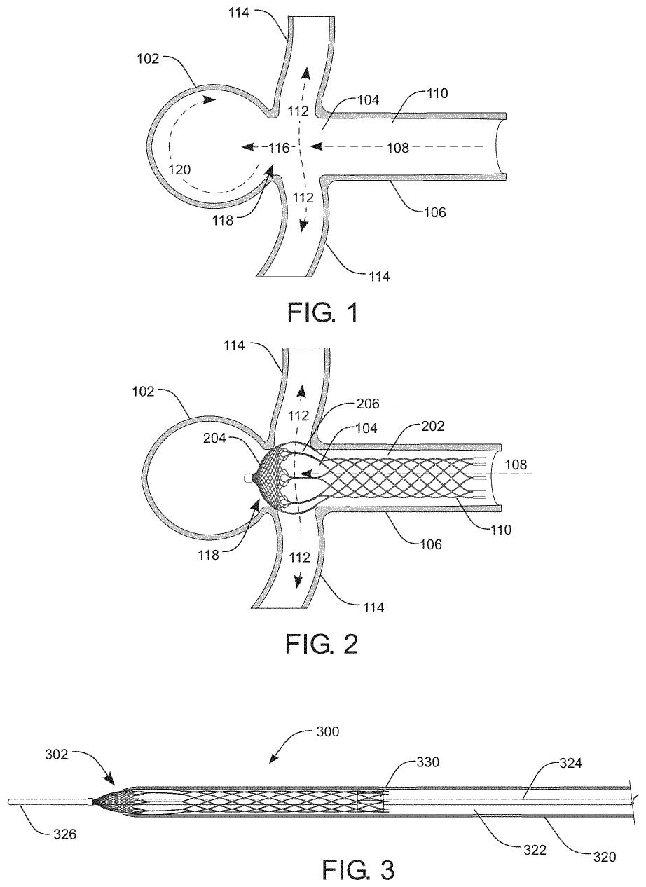

One type of aneurysm that can be challenging to effectively treat occurs at a bifurcation of a blood vessel into multiple secondary blood vessels. FIG. 1 shows such an aneurysm 102 at a bifurcation 104 of a blood vessel 106. Blood flows 108 through the lumen 110 of a primary blood vessel 106 and, in this example, splits to flow 112 through two secondary blood vessels 114. A portion 116 of the blood flow, however, flows into the aneurysm 102 through an aneurysm ostium 118 at the bifurcation 104. This portion 116 of the blood flow 108 can increase internal aneurism pressure and tends to circulate 120 within the aneurysm 102.

Various techniques exist to treat aneurysms at bifurcated blood vessels. One example of an invasive technique includes a surgical procedure involving placing a clip across the neck of the aneurysm to curtail blood from entering therein. An example of a minimally invasive technique involves placing a microcatheter within the aneurysm and deploying coils therein to cause thrombosis within the aneurysm to block blood flow. This technique, however, can puncture through the aneurysm wall, which leads to aneurysm rupture. In some cases, a portion of the coils can migrate out of the aneurysm and into the blood vessel, potentially causing damage to other blood vessels and/or neural tissue. Another example of a minimally invasive technique involves placing stents in the primary and secondary blood vessels to limit blood flowing into the aneurysm. Such a technique can be difficult to achieve and can significantly limit blood flow through the bifurcation of the blood vessel.

The present disclosure provides a minimally invasive technique of delivering and using a flow diverter that addresses many, if not all, of the aforementioned issues.

Additionally, examples of flow diverter delivery systems according to the present disclosure include a novel attachment and release mechanism that secures a flow diverter during delivery up to a nearly complete deployment, while maintaining the capacity to withdraw the flow diverter back into the catheter. Current delivery mechanisms generally utilize friction forces to hold implants in delivery devices during deployment. Once the friction forces are reduced to a point where the delivery mechanism can no longer maintain a grip on the implant, the implant is released. Furthermore, the point at which full deployment occurs with such mechanisms, such as friction pad delivery systems for example, can vary depending on the inherently inconsistent friction forces applied by the delivery mechanism to a given implant. The present flow diverter delivery systems, however, utilize a mechanical engagement to releasably secure a flow diverter prior to and during deployment. Such mechanical attachment allows the flow diverter to be deployed to a much greater extent compared to current delivery mechanisms, while maintaining the capacity to withdraw the flow diverter back into the catheter. Additionally, the point of full deployment of a flow diverter held by a mechanical attachment according to flow diverter delivery systems of the present disclosure, however, is predictable and consistent, thus allowing a medical professional to reliably know the point at which deployment will occur.

As one example, as is shown in FIG. 2, a flow diverter 202 is positioned within a blood vessel 106 at a bifurcation 104 between the blood vessel 106 and secondary blood vessels 114. The flow diverter 202 is longitudinally positioned against an ostium 118 of an aneurism 102 at the bifurcation 104. The flow diverter 202 is thus positioned relative to the ostium 118 to divert blood flow from the primary blood vessel to the secondary blood vessels, thereby reducing blood flow into the aneurism. The flow diverter 202 (i.e., linear implant) includes a low-porosity distal cap 204 (distal cap) having an outer convex shape that is structurally configured to be longitudinally positioned adjacent a luminal wall of a blood vessel bifurcation 104 at an aneurysm ostium 118. In some examples, the distal cap 204 of the flow diverter 202 can be inserted into or slightly within the aneurysm 102 against the lumen side of the blood vessel at the aneurysm ostium 118. Once in position, the distal cap 204 reduces blood flow 108 entering the aneurysm 102, which is diverted to flow 112 through the secondary blood vessels 114. In other words, the distal cap 204 diverts blood flow 108 to the secondary blood vessels 114, thereby reducing both blood flow 116 into, and pressure at, the aneurysm.

Without intending to be bound by any scientific theory, in one example the distal cap 204 can be sufficiently porous allow some blood flow therethrough to facilitate endothelization (as opposed to thrombosis) across the distal cap 204, which will further block blood flow 108 from entering the aneurism 102. This technique can significantly decrease the likelihood of rupture or other adverse cerebral events, thus significantly deceasing the severity of the health risk and improving the prognosis of the affected patient. It is noted that the depictions of the aneurysms and bifurcated blood vessels in FIGS. 1 and 2 are merely simplified examples and should not be seen as limiting.

Flow diverters of the present disclosure generally divert the flow of blood to the secondary blood vessels by reducing blood flow into the aneurysm from the blood vessel side of the bifurcation. Reducing such blood flow without the device being physically positioned within the lumen of the aneurysm significantly reduces the risk of aneurysm wall ruptures, which also results in a significantly improved prognosis for the patient.

A flow diverter delivery system is additionally provided, which can generally include a catheter (i.e., a delivery catheter) that is sized for insertion and movement through a blood vessel, a pusher wire slidably disposed within the catheter, a distal guide linearly coupled to a distal end of the pusher wire, a flow diverter anchor coupled to the distal guide, and a flow diverter having an undeployed configuration and a deployed configuration that is releasably coupled to the flow diverter anchor in the undeployed configuration. The flow diverter is mechanically coupled to the flow diverter anchor and is held in the undeployed configuration by the inside surface of delivery lumen constraining the flow diverters radial expansion. The flow diverter is mechanically coupled to the flow diverter anchor in such a way that radial expansion at the proximal end of the flow diverter releases the mechanical coupling. As such, the flow diverter can be retrieved back into the catheter prior to release of the mechanical coupling.

In a more specific example, as is shown in FIG. 3, a flow diverter delivery system 300 includes a catheter 320 having a delivery lumen 322, which can include any type of catheter or tubular delivery device that is sized for insertion and movement through a blood vessel. The delivery lumen 322 refers to the inner-most surface of the catheter 322, which can include the inside surface of the catheter or the inside surface of a sheath or other tubular structure within the catheter. The flow diverter delivery system further includes a pusher wire 324 slidably disposed within the delivery lumen 322 and a distal guide 326 linearly coupled to a distal end of the pusher wire 324. A flow diverter anchor 330 is coupled to the distal guide 326, the pusher wire 324, or both. A flow diverter 302 having an undeployed configuration and a deployed configuration is releasably coupled to the flow diverter anchor 330 when in the undeployed configuration. The flow diverter 302 is mechanically coupled to the flow diverter anchor 330 and is held in the undeployed configuration by the inside surface of delivery lumen 322 constraining the flow diverters radial expansion. The flow diverter 302 is mechanically coupled to the flow diverter anchor in such a way that radial expansion at the proximal end of the flow diverter releases the mechanical coupling. As such, the flow diverter can be retrieved back into the catheter prior to release of the mechanical coupling.

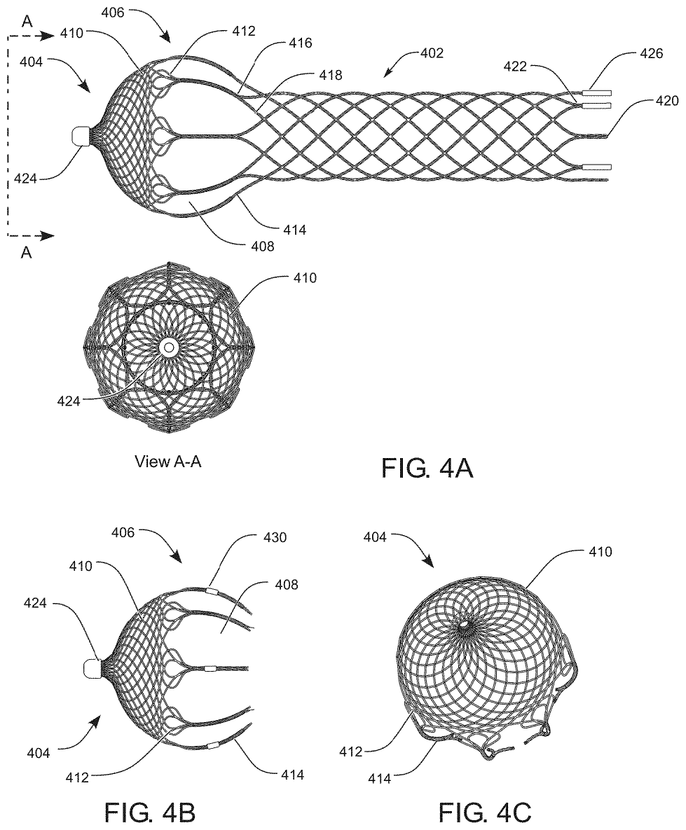

The flow diverter shown in FIG. 4A can be made of any useful material capable of achieving results as outlined herein. For example, the flow diverter can be made from laser cut materials, polymeric materials, carbon nanotubes, wire materials, braided wires, braided wire bundles, and the like, including combinations thereof. The distal cap can be made from a variety of materials, as is described below. In one example, the flow diverter is made from wires or wire bundles 410 that are braided together to, for example, form the distal cap 404 that allows the flexibility to design and make different portions of the flow diverter to have different physical properties and functionality when deployed and placed inside of a blood vessel.

In another nonlimiting example, shown in FIG. 4A, a flow diverter includes a linear support body 402, such as, for example, a support stent, a low-porosity distal cap 404, and transverse flow section 406 having multiple transverse openings 408 between the linear support body 402 and the low porosity distal cap 404. In the nonlimiting example shown in FIG. 4A, the low-porosity distal cap 404 is made of wires 410 that are braided into a pattern (View A-A) extending from a distal wire attachment 424. The wires 410 can be braided according to any useful pattern that allows the flow diverter to be deployed and that is sufficiently stiff to hold the transverse flow section 406 and the distal cap 404 in position at the aneurysm ostium. Wires 410 making up the braided pattern of the low-porosity distal cap 404 can be single wires or multiple wires, braided or otherwise associated together, depending on the design of the device. At the proximal end of the low-porosity distal cap 404, the wires 410 weave together to form a plurality of wire slack adjusters 412, from which the wires 410 from each wire slack adjuster 412 gather together to form a primary braided wire bundles 414. Each primary braided wire bundle 414 branches proximally at divergence point 416 to form multiple secondary braided wire bundles 418, which are then braided together to form the linear support body 402.

The wires 410 (or braided wire bundles) of the secondary wire bundles 418 can terminate at the proximal end of the linear support body 402 according to a variety of techniques and/or structures, which can depend, at least in part, on the design characteristics of the diverter device. In one example, the proximal end of the linear support body 402 includes multiple termination wire bundles 420, where each termination wire bundle 420 includes the wires 410 from at least two secondary wire bundles 418 coupled together at convergence point 422. It is additionally contemplated that each secondary wire bundle can be woven throughout the linear support body 402 without converging with another secondary wire bundle 418, except, in some examples, at the convergent point 422. The wires 410 of the termination wire bundles 420 can be secured together to at least maintain the integrity of the linear support body 402. In one example, the wires of each termination wire bundle can be secured together by fusing, such as by soldering or electrically welding. In another example, a binder material can be applied thereto, such as through electrolytic deposition, polymeric coating, or the like, among other things. In yet another example, at least a portion of the wires of the termination wire bundles are crimped together using a wire bundle clip. In some cases, the wire bundle clip is radio-opaque, which can allow the termination wire bundles to be imaged during an implantation procedure. Other structures can optionally be made from radio-opaque materials to facilitate imaging, including the distal wire attachment or one or more wires woven through the device, without limitation.

FIG. 4B shows an example of the distal cap 404 and the transverse flow section 406 having multiple transverse openings 408. The proximal end of the distal cap 402 transitions into the plurality of wire slack adjusters 412, from which the wires 410 from each wire slack adjuster 412 gather together to form a primary braided wire bundle 414. One or more wire clips 430, optionally made from a radio-opaque material, are crimped around one or more primary wire bundles 414. FIG. 4C shows a view of the distal cap 404 from the inside of the flow diverter that includes the wires 410, the wire slack adjustors, and the primary wire bundles 414.

More generally, however, in one example the distal cap can be made of braded wire. In another example, the distal cap can be made of a laser cut material. Similarly, each of the distal cap coupling, the transverse flow section, the distal support body coupling, and the linear support body can be independently made from braided wire, laser cut material, or a combination thereof.

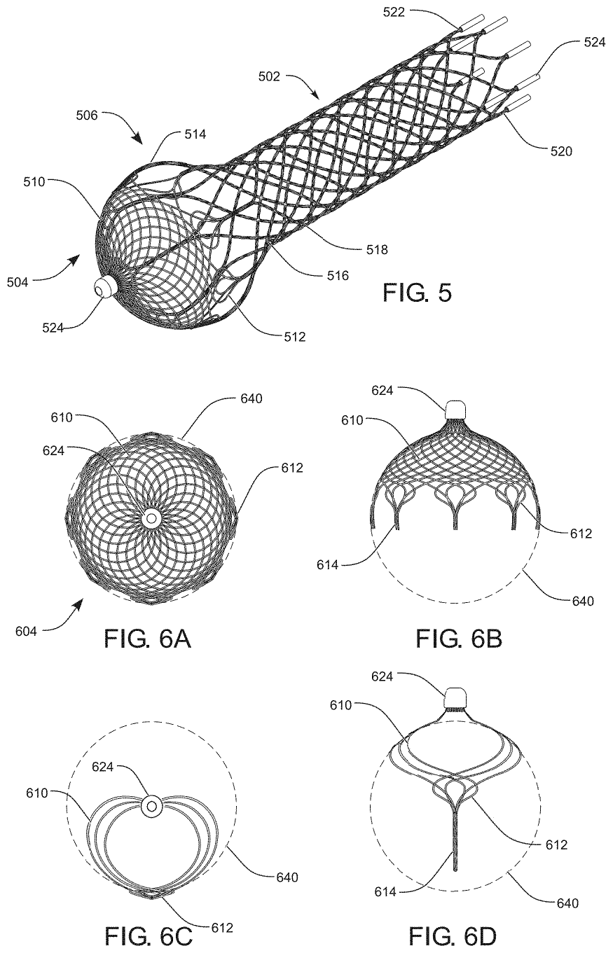

FIG. 5 shows an isometric view of a flow diverter including a linear support body 502, such as, for example, a support stent, a low-porosity distal cap 504, and transverse flow section 506 having multiple transverse openings 508 between the linear support body 502 and the distal cap 504. In the nonlimiting example shown in FIG. 5, the distal cap 504 is made of wires 510 that are braided into a pattern extending from a distal wire attachment 524. The wires 510 can be braided according to any useful pattern that allows the flow diverter to be deployed and that is sufficiently stiff to hold the transverse flow section 506 and the distal cap 504 in position at the aneurysm ostium. Wires 510 making up the braided pattern of the low-porosity distal cap 504 can be single wires or multiple wires, braided or otherwise associated together, depending on the design of the device. At the proximal end of the distal cap 504, the wires 510 weave together to form a plurality of wire slack adjusters 512, from which the wires 510 from each wire slack adjuster 512 gather together to form a primary braided wire bundles 514. Each primary braided wire bundle 514 branches proximally at divergence point 516 to form multiple secondary braided wire bundles 518, which are then braided together to form the linear support body 502.

FIG. 6A shows an example view of a distal cap 604 looking proximally from the distal wire attachment 624. FIG. 6B shows an example side view of the distal cap 604. The distal cap 604 includes a plurality of wires 610 that are braided into a pattern from the distal wire attachment 624. In one nonlimiting example, the wires 610 can be braided over a sphere 640 to form the distal cap 604 according to any useful pattern that is deployable and that is sufficiently stiff to hold the distal cap 604 in position at the aneurysm ostium. Wires 610 of the distal cap 604 can be single wires or multiple wires, braided or otherwise associated together, depending on the design of the device. FIGS. 6C and 6D show the views from FIGS. 6A and 6B with only the wires 610 associated with a single wire slack adjuster 612, for clarity.

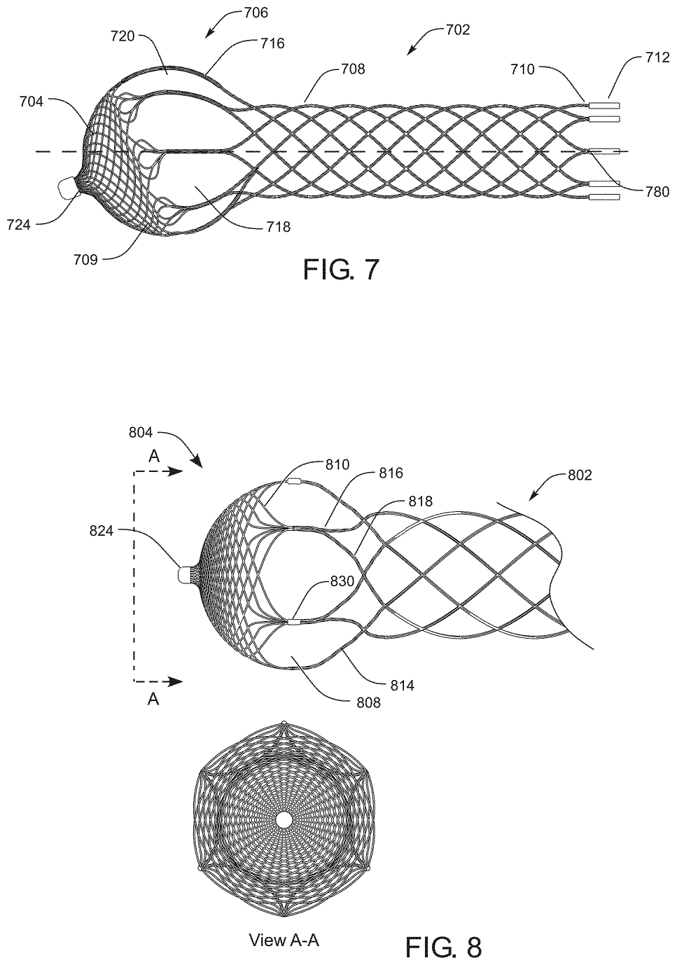

FIG. 7 shows a nonlimiting example of a flow diverter including a linear support body 702, such as, for example, a support stent, a low-porosity distal cap 704, and transverse flow section 706 having multiple transverse openings 708 between the linear support body 702 and the distal cap 704. The distal cap 704 is made of wires 710 that are braided into a pattern extending from a distal wire attachment 724. The wires 710 can be braided according to any useful pattern that allows the flow diverter to be deployed and that is sufficiently stiff to hold the transverse flow section 706 and the distal cap 704 in position at the aneurysm ostium. Wires 710 making up the braided pattern of the low-porosity distal cap 704 can be single wires or multiple wires, braided or otherwise associated together, depending on the design of the device. At the proximal end of the distal cap 704, the wires 710 weave together to form a plurality of wire slack adjusters 712, from which the wires 710 from each wire slack adjuster 712 gather together to form a primary braided wire bundles 714. Each primary braided wire bundle 714 branches proximally at divergence point 716 to form multiple secondary braided wire bundles 718, which are then braided together to form the linear support body 702. In the example shown in FIG. 7, the distal wire attachment 724 and the surrounding weave of the braided wires 709 are offset or otherwise rotated away from the central axis 780 of the flow diverter. In this case, the center of the dense portion of the distal cap 704 is not positioned to align along the linear central axis of the linear support body 702. As such, flow diverters can be made that have orientations/configurations that more closely approximate the orientations/configurations of a given aneurysm/bifurcation.

FIG. 8 shows another example of a device having wires 810 braided into a pattern that forms a low-porosity distal cap 804. The wire bundle 816 can be secured or otherwise coupled or held together by any technique known to those skilled in the art. All of the braided wire in a device can be secured by the same mechanism or different mechanisms. The example in FIG. 8 shows braided wire 816 having different securing mechanisms. Certain wire bundles 816 are secured together with wire clips 830 that crimp each wire bundle 816 securely together. Other wire bundles 816 lack wire clips and can be secured together with any technique capable of securing such wire bundles together. In one example, the wires of each wire bundle can be twisted or woven together. In another example, the wire bundles can be secured together by heat treatment. In yet another example, the wire bundles can be secured together with a bonding material. It is additionally contemplated that the wire bundles 816 can be left unsecured.

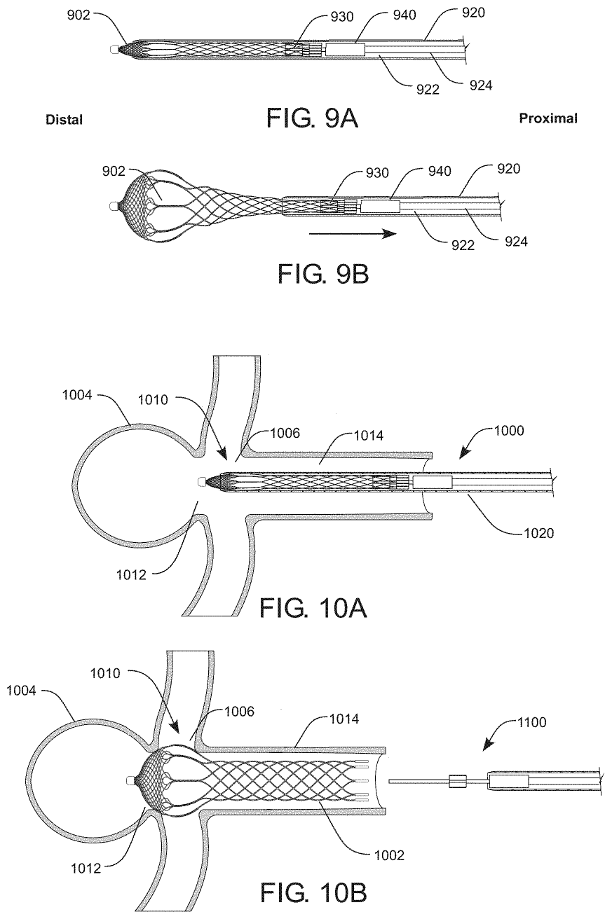

In another example, the present disclosure provides a system for delivering a flow diverter, as is shown in FIG. 9A. Such a system can include a catheter 920 having a delivery lumen 922, a pusher wire 924 slidably disposed within the delivery lumen 922 and a distal guide (not shown for clarity) linearly coupled to a distal end of the pusher wire 924. A flow diverter anchor 930 is coupled to the distal guide, the pusher wire 924, or both, to which a flow diverter 902 having an undeployed configuration and a deployed configuration is releasably coupled when in the undeployed configuration. As is shown in FIG. 9B, when the catheter 920 is withdrawn (see arrow) from the distal end of the flow diverter 902 (or when pusher wire 924 is pushed distally, or both), the flow diverter begins to deploy into the deployed configuration.

FIGS. 10A and 10B show the placement and delivery of the flow diverter 1002 from the flow diverter system 1000 at an aneurysm 1004 at a bifurcated blood vessel 1006. As is shown in FIG. 10A, the flow diverter system is passed through the lumen of the blood vessel 1014 and the distal end 1010 of the flow diverter system 1000 is positioned at or near the aneurysm ostium 1012. As is shown in FIG. 10B, the catheter 1020 of the flow diverter system 1000 is pulled back to expose the flow diverter 1002, the flow diverter 1002 is deployed and released at the aneurysm ostium 1012 and the remaining portion 1100 of the flow diverter delivery system is withdrawn.

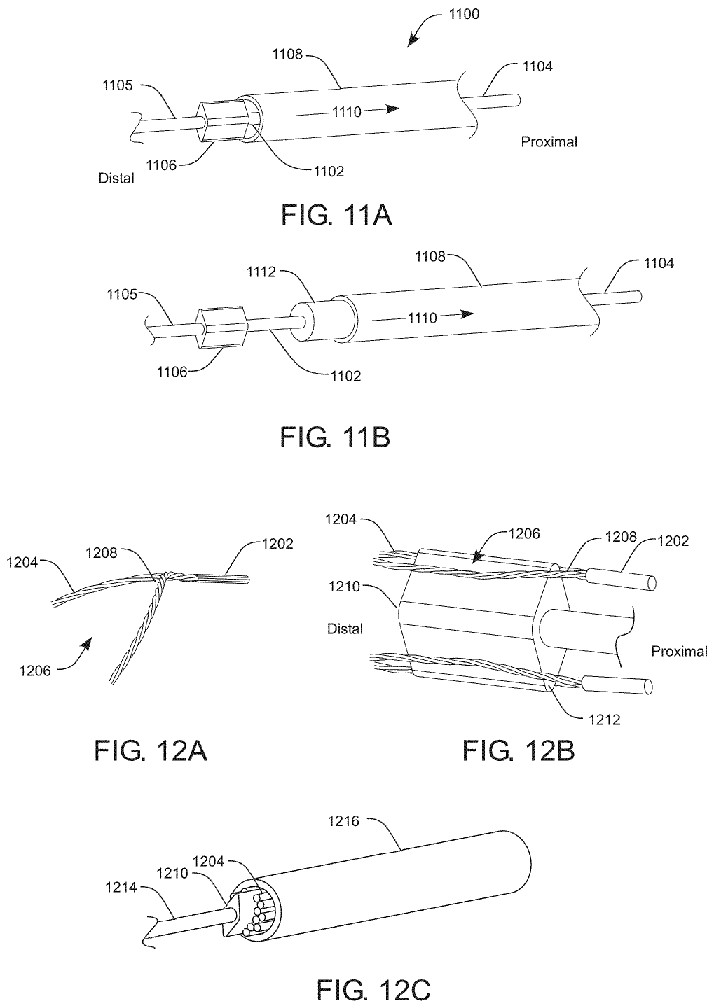

FIGS. 11A and 11B show an example of a delivery device capable of deploying and retrieving a flow diverter. FIG. 11A shows a delivery device 1100 having a spacer 1102, a pusher wire 1104, and a distal guide 1105. In some examples, either the spacer 1102 or the spacer 1102 and the distal guide 1105 are a continuous extension of the pusher wire 1103. In other examples, either the distal guide 1105 or the distal guide and the spacer 1102 are separate components from the pusher wire 1104, either of the same material or different materials. A flow diverter anchor 1106 is positioned between the spacer 1102 and the distal guide 1105, which is configured to couple to the proximal end of a flow diverter (not shown). A catheter 1108 initially encloses the spacer 1102, the distal guide 1105, and the flow diverter anchor 1106 prior to deployment of the flow diverter. When the flow diverter is enclosed by the catheter 1108, the flow diverter is compressed into an undeployed configuration. As the catheter 1108 moves in a direction shown by arrow 1110, the flow diverter begins to deploy as it is released from the confinement of the catheter 1108. The flow diverter does not fully deploy until the catheter 1108 has been sufficiently withdrawn to allow flow diverter anchor 1106 to release the proximal end of the flow diverter. Prior to reaching this “point of no return,” the flow diverter can be pulled back into the catheter 1108. FIG. 11B shows an example of a flow diverter delivery system showing the withdrawn catheter 1108 with the spacer 1102, the distal guide 1105, and the flow diverter anchor 1106, exposed to a greater extent than what would be required to fully deploy the flow diverter for clarity. FIG. 11B also shows a pusher coupling 1112 to couple to the spacer 1102 to the pusher wire 1104.

In some examples, one or more components of a flow diverter delivery system can be made from a radiopaque material to allow the flow diverter delivery system to be imaged during the flow diverter procedure. Such real time visualization allows the medical professional to guide the flow diverter delivery system through the blood vessel to a target location. Furthermore, once reaching the target location, the flow diverter can be more accurately positioned as a result of such visualization.

In some examples, flow diverters can be made from braided wires, as is described more fully below. In other examples, flow diverters can be made using a cutting process, such as laser cutting to form laser-cut flow diverters. In both cases, a series of transverse openings is formed in the sides of the flow diverter, as can be seen in FIGS. 10A and 10B, for example. When the flow diverter is in a compressed or a partially deployed state, the flow diverter anchor engages one or more transverse openings to form a mechanical attachment or engagement that secures the flow diverter to the flow diverter anchor and allows retraction of the flow diverter into the catheter up to the point of full deployment.

Various flow diverter anchor designs can be utilized to form a mechanical attachment with transverse openings of a flow diverter. In one example, a flow diverter anchor includes a proximal transverse edge that is structurally configured to mechanically engage a distal-facing region or edge of a proximal transverse opening when a flow diverter is in an undeployed configuration. FIG. 12A shows an example of a proximal termination 1202 of a braided wire flow diverter where bundles of wires 1204 converge, thus forming a transverse opening 1206 having a distal-facing region 1208, in this case at a proximal end of the flow diverter. As such, a plurality of transverse openings can thus be formed around the periphery of the proximal end of the flow diverter. FIG. 12B shows an example of a flow diverter anchor 1210 having a proximal transverse edge 1212, in this case a proximal-facing proximal transverse edge. The proximal transverse edge 1212 mechanically engages engaging an opening 1206, in this case two openings 1206, formed by two pairs of braided wire bundles 1204 as they merge into two proximal terminations 1202. While in a compressed or partially deployed state, the overlying catheter (not shown) maintains the mechanical attachment between the proximal transverse edge 1212 of the flow diverter anchor 1210 and the distal-facing region 1208 of the transverse opening 1206 by limiting the radial movement of the distal-facing region 1208 of the transverse opening 1206 away from the transverse edge 1212, which would release the mechanical attachment. Once the catheter is sufficiently withdrawn to allow the distal-facing region 1208 of the transverse opening 1206 to expand or move radially from the transverse edge 1212, the flow diverter is released from the flow diverter anchor 1210 and allowed to fully deploy. It is noted that, while FIGS. 12A and 12B show the flow diverter anchor mechanically attaching to the openings at the proximal terminations of the flow diverter, any location along the flow diverter capable of receiving and forming a mechanical attachment with the flow diverter anchor can be similarly utilized and is considered to be within the present scope.

FIG. 12B shows an example of a flow diverter anchor 1210 coupled to a distal guide 1214. The flow diverter anchor 1210 is shown protruding from a catheter 1216, with a plurality of braided wire bundles 1204 from a flow diverter surrounding the flow diverter anchor 1210. The braided wire bundles 1204 couple together proximally to form transverse openings (not shown), at least one of which is held in a mechanically locked configuration with the flow diverter anchor 1210 by an inner wall of the catheter 1216, thus maintaining the flow diverter compressed into an undeployed state. When the braided wire bundles 1204 are released from the confinement of the catheter 1216, the flow diverter is allowed to expand, transverse opening and the flow diverter anchor 1210 are released from the mechanically locked configuration, and the flow diverter is released from the flow diverter anchor 1210. FIG. 12C shows an isometric view of an example of a flow diverter anchor 1210 and a distal guide 1214 protruding from a catheter 1216, with a plurality of braided wire bundles 1204 from a flow diverter surrounding the flow diverter anchor 1210.

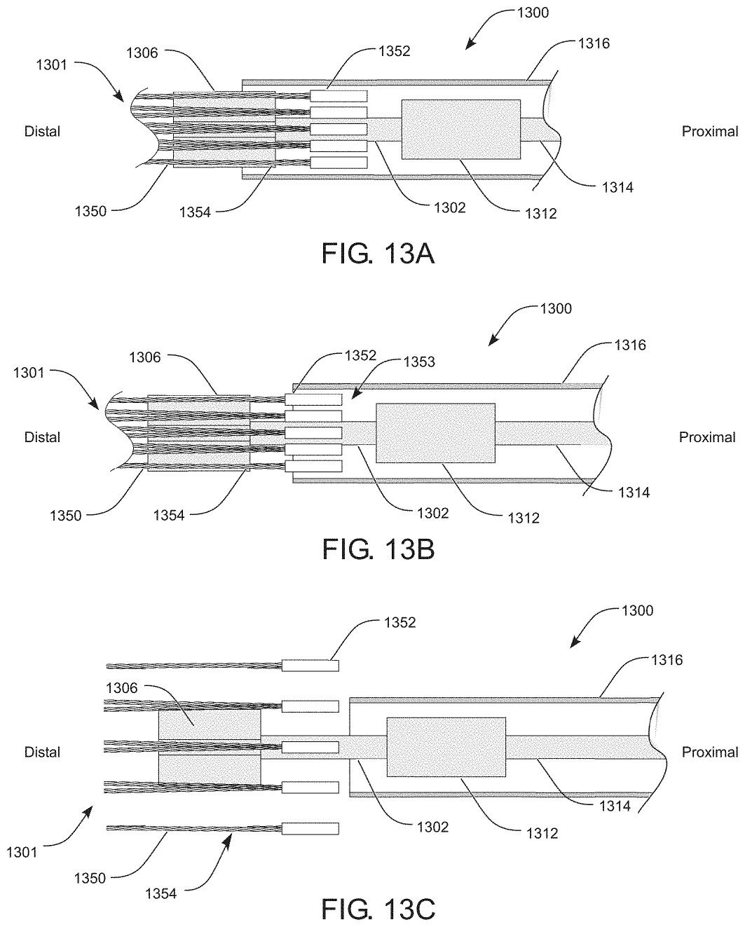

FIG. 13A shows a flow diverter delivery system 1300 mechanically coupled to a wire-braided flow diverter 1301. The flow diverter delivery system includes a spacer 1302 coupled between a flow diverter anchor 1306 and a pusher coupling 1312. The pusher coupling 1312 provides an engagement between the spacer 1302 and a pusher wire 1314. The flow diverter 1301 includes a plurality of braided wire bundles 1350 that form multiple transverse openings 1354 as they converge into multiple proximal terminations 1352. When the flow diverter 1301 is in an undeployed state, as is shown in FIG. 13A, the flow diverter anchor 1306 engages one or more transverse openings 1354 to form a mechanical attachment that secures the flow diverter 1301 to the flow diverter delivery system 1300. The flow diverter 1301 is prevented from deploying by an overlying catheter 1316 that limits radial movement of the braided wire bundles 1350 away from the spacer 1302. As such, the flow diverter 1301 is secured to the flow diverter delivery system 1300 by the mechanical engagement between the transverse openings 1354 and the flow diverter anchor 1306 until the proximal end of the flow diverter 1301, in this case the proximal terminations 1352, is released from the catheter 1316.

FIG. 13B shows the catheter 1316 pulled back (or the pusher wire 1314 moved forward, or both) to expose a distal portion of the proximal terminations 1352. At this point the transverse openings 1354 remain mechanically engaged with the flow diverter anchor 1306, thus maintaining the capacity for the flow diverter 1301 to be fully or partially withdrawn into the catheter 1316. This capacity is maintained until the proximal end 1353 of the flow diverter 1301 has been exposed from the catheter 1316 to a degree that allows the proximal terminations 1352 sufficient radial movement such that the transverse openings 1354 disengage from the flow diverter anchor 1306. Depending on the distance of the openings 1354 from the proximal terminations 1352, disengagement can occur as the proximal terminations 1352 are released from the catheter 1316 or prior to the release of the proximal terminations 1352 from the catheter 1316.

FIG. 13C shows the release of the flow diverter 1301 from the flow diverter anchor 1306 of the flow diverter delivery system 1300 and into the fully deployed state. As the catheter 1316 releases the proximal terminations 152, the body of the flow diverter 1301 expands radially away from the flow diverter anchor 1306, thus breaking the mechanical attachment therewith.

In one example, the proximal transverse edge of the flow diverter anchor is structurally configured to mechanically disengage from the proximal transverse opening when the delivery lumen is withdrawn to fully expose a proximal end of the flow diverter. In another example, the proximal transverse edge of the flow diverter anchor is structurally configured to mechanically disengage from the proximal transverse opening when the delivery lumen is withdrawn to expose a proximal end of the flow diverter sufficiently to allow the proximal transverse opening to lift off of the proximal transverse edge between the flow diverter anchor and the delivery lumen.

The flow diverter can thus be deployed to a significant extent while retaining the capacity to withdraw the flow diverter back into the catheter. In one example, the proximal transverse edge of the flow diverter anchor and the delivery lumen are structurally configured to maintain the capacity to withdraw the flow diverter into the delivery lumen when the flow diverter is at least 70% deployed. In another example, the proximal transverse edge of the flow diverter anchor and the delivery lumen are structurally configured to maintain the capacity to withdraw the flow diverter into the delivery lumen when the flow diverter is at least 80% deployed. In a further example, the proximal transverse edge of the flow diverter anchor and the delivery lumen are structurally configured to maintain the capacity to withdraw the flow diverter into the delivery lumen when the flow diverter is at least 90% deployed.

It is noted that the presently described mechanical attachment functions according to passive release, whereby the withdrawal of the catheter releases the mechanical engagement by allowing the openings of the braided wire bundles to expand away from the flow diverter anchor. It is additionally noted that the flow diverter expansion can include self-expansion or expansion by other mechanical mechanisms, such as balloon assisted expansion. The flow diverter anchor can be formed into a variety of shapes and sizes and can attach to one or more openings in the flow diverter, including two or more openings.

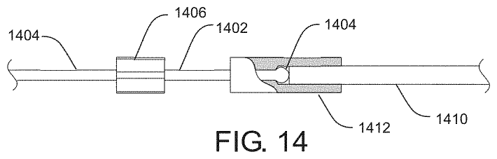

FIG. 14 shows one example cross section of a flow diverter delivery system including a spacer 1402 a distal guide 1404. A flow diverter anchor 1406 is positioned between the spacer 1402 and the distal guide 1404, which is configured to couple to the proximal end of a flow diverter (not shown). A pusher coupling 1412 provides an attachment point 1404 between the spacer 1402 and a pusher wire 1410.

The flow diverter delivery systems of the present disclosure can be made from various materials, as is known to those of ordinary skill in the art. For example, pushers, pusher couplings, spacers, flow diverter anchors, and the like can be made from any physiologically compatible material that has appropriate material characteristics to perform delivery and deployment of a flow diverter as outlined herein. Nonlimiting examples of such materials can include nitinol materials, stainless steel, platinum, titanium, iridium, etc., including alloys and mixtures thereof.

Radiopaque materials used in the presently disclosed devices can be any biologically compatible material capable of being incorporated therein. Nonlimiting examples of radiopaque materials can include tantalum, tungsten, bismuth, gold, titanium, platinum, palladium, rhodium, iridium, tin, and mixtures, blends, composites, and alloys thereof.

Flow diverters can be made from a variety of materials known to those of ordinary skill in the art. For example, a flow diverter can be made from laser cut materials, polymeric materials, fabric materials, braided wire materials, and the like. In one example, a flow diverter is made from bundles of wires braided together. In another example of the present disclosure, a flow diverter can be made from mixed materials, or in other words, a combination of two or more laser cut materials, polymeric materials, wire materials, braided wire materials, and the like, including any other materials known to those skilled in the art that can be beneficially used in the presently disclosed devices. Furthermore, wire used to create wire bundles can be any physiologically compatible memory alloy capable of forming a flow diverter as per the present disclosure. Nonlimiting examples of shape memory alloys can include Ag—Cd, Au—Cd, Co—Ni—Al, Co—Ni—Ga, Cu—Al—Be—X (where X is Zr, B, Cr, or Gd), Cu—Al—Ni, Cu—Al—Ni—Hf, Cu—Sn, Cu—Zn, Cu—Zn—X (where X is Si, Al, or Sn), Fe—Mn—Si, Fe—Pt, Mn—Cu, Ni—Fe—Ga, Ni—Ti, Ni—Ti—Hf, Ni—Ti—Pd, Ni—Mn—Ga, Ti—Cr or Ti—Nb, including combinations thereof. In another example, the wire can include a drawn filled tubing wire. While any combination of useful wire materials is contemplated, in one example the outer tube can be made of a nickel/titanium alloy and the inner core material can be a radiopaque material.

In one specific nonlimiting example, a metal alloy of nickel and titanium (Nitinol®) can be used as wires used to create the braided wire. Nitinol alloys are named according to the weight percentage of nickel in the alloy. For example, Nitinol 50, Nitinol 55, and Nitinol 60 include weight percentages of nickel in the alloy of 50%, 55%, and 60%, respectively. Any alloy of Nitinol can be used in the wire bundles that can be used to make a flow diverter according to the present disclosure. Furthermore, the diameter of the Nitinol wire (or any other shape memory alloy wire) can be from about 0.008 inches to about 0.0005 inches in diameter in one example, from about 0.005 inches to about 0.0009 inches in diameter in another example, and from about 0.002 inches to about 0.0015 inches in diameter, without limitation.

In one example the degree of porosity of the distal cap can play a role in successfully diverting blood flow from an aneurysm over the long-term. If the porosity of the distal cap is sufficiently low to block blood flow to a degree that thrombosis is facilitated on the aneurysm side of the distal cap, the growing thrombus can spread through the periphery of the ostium of the aneurysm and across the structure of the flow diverter. Such a thrombus can cause further complications to the patient that can, in some cases, be life-threatening. A higher porosity that diverts blood flow to the secondary blood vessels but which allows sufficient blood flow therethrough to facilitate fibrosis, endothelization, or delayed thrombosis across the distal cap and the ostium of the aneurysm can result in a successful flow diverter placement with significantly reduced complications. In terms of the flow diverter, porosity is merely the inverse of the wire density of the distal cap. Such can additionally be described as coverage when referring to the inverse of the porosity of the ostium with the flow diverter in place (i.e., metal coverage for metal wires, polymer coverage for polymeric wires, etc.). One skilled in the art can readily ascertain a proper porosity/density of the distal cap to achieve such a result, once in possession of the present disclosure. In one example, however, the density of the distal cap can be from about 40% to about 85% or from about 50% to about 70%. In some examples the porosity of the distal cap can be from about 15% to about 55%, from about 15% to about 60%, from about 30% to about 50%, or from about 25% to about 45%. Furthermore, in some examples, the density of the braided wires in the distal cap can vary from the center to the periphery. For example, the density can be highest at the center of the distal cap where the braided wires couple to the distal wire attachment and lower at the periphery adjacent the transverse flow section. In one example, without limitation, the change from a higher density at the center of the distal cap to a lower density at the periphery of the distal cap can be a uniform transition. In another example, without limitation, the change from a higher density at the center of the distal cap to a lower density at the periphery of the distal cap can be a nonuniform transition.

The porosity of the distal cap can be determined by the number, the diameter, and/or the weave pattern of the wires used in the device. As such, the number of wires and the number of wires in the wire bundles can vary, depending on the design and desired properties of the device. For example, the number of wires in a flow diverter can be multiples of 3, 4, 5, 6, 7, 8, and so on, provided that the proper porosity of the resulting distal cap can function as outlined herein. In one specific case, however, the number of wires is a multiple of 6, for example, 24 wires, 36 wires, or 48 wires, without limitation. As such, flow diverters would have 6 bundles of 4 wires or 4 bundles of 6 wires, 6 bundles of 6 wires, or 6 bundles of 8 wires or 8 bundles of 6 wires, respectively. As such, any weave pattern can be used that, taking into account the number of wires and wire bundles used, can be woven into a distal cap having a uniform or nonuniform density as described and the desired density/porosity as understood by one skilled in the art. Furthermore, in one example, all of the wires can be the same length. In another example, at least a portion of the wires can have different lengths.

In one specific nonlimiting example, a metal alloy of nickel and titanium (Nitinol®) can be used as wires used to create the braided wire. Nitinol alloys are named according to the weight percentage of nickel in the alloy. For example, Nitinol 50, Nitinol 55, and Nitinol 60 include weight percentages of nickel in the alloy of 50%, 55%, and 60%, respectively. Any alloy of Nitinol can be used in the wire bundles that can be used to make a flow diverter according to the present disclosure. Furthermore, the diameter of the Nitinol wire (or any other shape memory alloy wire) can be from about 0.008 inches to about 0.0005 inches in diameter in one example, from about 0.005 inches to about 0.0009 inches in diameter in another example, and from about 0.002 inches to about 0.0015 inches in diameter, without limitation.

The linear support body can have any weaving pattern of wire bundles, provided the linear support body has sufficient longitudinal strength/stiffness to hold the distal cap in position at the aneurysm ostium with sufficient radial force at the distal cap to keep it in contact with the inner aneurysm ostium. Additionally, where a wire bundle crosses over other wire bundles, they can be woven in an over/under pattern, in one example. In other examples, the wire bundle can be woven in other patterns, such as two over one under and the like. Furthermore, in one example the wires in the wire bundles can be twisted around one another. In another example, the wires can be positioned side-by-side with little to no twisting. In another example, the wires can be positioned side-by-side with little to no twisting in certain locations along the linear support body and twisted around one another in other sections. The same twisting examples can apply for the transverse flow section and the distal cap.

For a Nitinol wire stent (or linear support body), the wire bundles can be heat treated such that the flow diverter achieves a desired configuration once deployed at the aneurism ostium, or in other words, the flow diverter rebounds to a fully expanded, deployed state. Additionally, such heat treatment can place the flow diverter in a deployed position that matches a certain type or positioning of the aneurysm ostium relative to the primary blood vessel.

The distal wire attachment and the proximal wire attachment can be made from any useful physiologically compatible material capable of coupling to the wires of the braided wire bundles. In some examples, the distal wire attachment and/or the proximal wire attachment can be made of a radiopaque material to enhance visualization of the flow diverter when in use. Furthermore, the wire clips that crimp together certain of the braided wire bundles can additionally be made of a radiopaque material in order to enhance visualization of the flow diverter section of the flow diverter. The radiopaque material used for the proximal wire attachment, the distal wire attachment, and/or the wire clips can be any physiologically compatible material capable of coupling to the wires or wire bundles as per the present disclosure. Nonlimiting examples of radiopaque materials can include tantalum, tungsten, bismuth, gold, titanium, platinum, palladium, rhodium, iridium, tin, and mixtures, blends, composites, and alloys thereof. In another example, the proximal wire attachment, the distal wire attachment, and/or the wire clips can be made of a nonradiopaque material. In such cases, one or more radiopaque marker(s) can be coupled to the flow diverter to allow visualization during placement.

In yet another example, the proximal wire attachment can additionally be utilized as a retriever for the flow diverter. As the wires of the wire bundles are coupled to the proximal wire attachment, by pulling the proximal wire attachment back toward a delivery catheter, the wire bundles can fold back into the deliver catheter and the flow diverter can be retrieved or partially retrieved. For example, the flow diverter can be retrieved or partially retrieved for repositioning at the aneurysm ostium.

### EXAMPLES

The present disclosure provides, in one example, a flow diverter including a linear device body having an undeployed configuration, a partially deployed configuration, and a deployed configuration, where the linear device body is sufficiently flexible to move through blood vessels in the undeployed configuration. When in the deployed configuration, the linear device body can further include a low-porosity distal cap having an outer convex shape structurally configured to be positionable adjacent or slightly within an ostium of an aneurysm at a blood vessel bifurcation, such that the distal cap is configured to divert at least a portion of blood flow from flowing into the aneurysm from the blood vessel bifurcation, a transverse flow section adjacent the distal cap structurally configured to allow blood flow through blood vessel bifurcation, and a linear support body adjacent the low-density section and structurally configured to stabilize the linear device body in a lumen of the blood vessel bifurcation.

In another example, the transverse flow section of the flow diverter includes a plurality of transverse openings.

In another example, the distal cap has a lower porosity compared to the linear support body and the transverse flow section has a higher porosity compared to the linear support body.

In another example, the distal cap has a porosity that allows sufficient blood flow into the aneurysm to inhibit thrombosis from forming on the distal cap and that restricts sufficient blood flow into the aneurism to facilitate endothelization on the distal cap.

In another example, the distal cap has a porosity of from about 15% to about 55%.

In another example, the distal cap has a porosity of from about 30% to about 40%.

In another example, the distal cap and the transverse flow section are comprised of braided wire and the linear support body is a laser cut linear support body.

In another example, the distal cap. the transverse flow section, and the linear support body are comprised of braided wire.

In another example, the linear device body can additionally include a plurality of proximal wire attachments at the proximal end of the linear support body, wherein the distal cap, the transverse flow section, and the linear support body are substantially constructed of braided wire terminally coupled at the plurality of proximal wire attachments.

In another example, the flow diverter includes a distal wire attachment coupled to distal ends of the braided wire and aligned along a central axis of the linear device body when in the deployed configuration.

In another example, the distal wire attachment includes a radiopaque material as a radiopaque distal marker.

In another example, the proximal wire attachment includes a radiopaque material as a proximal marker.

In another example, the braided wire is a shape memory braided wire.

In another example, the braided wire includes a nickel alloy.

In another example, the braided wire is a drawn filled tubing wire.

In another example, wherein each wire of the braided wire is the substantially same length.

In another example, a weave pattern of the braided wire increases in density from the periphery of the distal cap to the distal wire attachment.

In another example, a plurality of wire slack adjusters couple between the proximal end of the flow diverter distal cap and the distal end of the transverse flow section.

In another example, each of the plurality of wire slack adjusters is structurally configured to provide sufficient slack to allow each associated wire to stretch out along the central axis of the flow diverter in the undeployed configuration and to then take up sufficient slack to allow the flow diverter to deploy into its original shape in the deployed configuration.

In another example, each of the plurality of wire slack adjusters, at its distal end, transitions to a primary wire bundle of a plurality of primary wire bundles that form the transverse flow section.

In another example, each of the plurality of wire bundles splits into multiple secondary wire bundles of a plurality of secondary wire bundles, wherein the plurality of secondary wire bundles is braided into a pattern to form the linear support body.

In another example, the distal wire attachment is aligned along a central axis of the linear device body when in the deployed configuration.

In another example, the distal wire attachment is not aligned along a central axis of the linear device body when in the deployed configuration.

In another example, the distal wire attachment has a central opening configured to allow passage of a wire from an inside region of the distal cap to an outside region of the distal cap.

The present disclosure provides a method for diverting blood flow from an aneurysm through a blood vessel bifurcation. Such an example can include, positioning a delivery catheter containing the flow diverter at an aneurysm of the blood vessel bifurcation, removing the delivery catheter from the flow diverter to transition the flow diverter from the undeployed configuration to the deployed configuration, such that the distal cap of the flow diverter is positioned at an ostium of the aneurysm.

In another example, the flow diverter is configured to be repositioned to align the distal cap with the ostium of the aneurysm, either during the transition from the undeployed configuration to the deployed configuration, following the transition from the undeployed configuration to the deployed configuration, or following an at least partial retraction of the flow diverter from a partially deployed configuration into the delivery catheter.

The present disclosure provides, in one example, a delivery system for diverting blood flow from an aneurysm ostium at a blood vessel bifurcation, comprising a delivery catheter including a flow diverter contained therein, the delivery system configured to move through a system of blood vessels to a blood vessel bifurcation having an aneurysm and a flow diverter delivery system releasably coupled to flow diverter positioned in the lumen of the flow diverter delivery system, the flow diverter delivery system configured to maintain a position of the flow diverter as the flow diverter delivery system is removed from the flow diverter.

The present disclosure provides, in one example, a flow diverter delivery system including a catheter having a delivery lumen and sized for insertion and movement through a blood vessel, a pusher wire slidably disposed within the delivery lumen, a distal guide linearly coupled to a distal end of the pusher wire, and a flow diverter having an undeployed configuration and a deployed configuration. The flow diverter, when in the deployed configuration, includes a low-porosity distal cap having an outer convex shape structurally configured to be longitudinally positionable adjacent a luminal wall of a blood vessel bifurcation at an aneurysm, a transverse flow section coupled to a proximal end of the distal cap, and a linear support body coupled to a proximal end of the transverse flow section and having a proximal transverse opening with a distal facing region, wherein the low-porosity distal cap is structurally configured to divert at least a portion of blood received from the linear support body through the transverse flow section. The flow diverter delivery system further includes a flow diverter anchor coupled to the distal guide and having a proximal transverse edge, the proximal transverse edge mechanically engaged to the distal-facing region of the proximal transverse opening of the flow diverter in the undeployed configuration, wherein the proximal transverse edge is further structurally configured such that radial movement of the proximal transverse opening away from the transverse edge disengages the flow diverter from the flow diverter anchor.

In another example, the transverse flow section includes a plurality of support members coupled between the distal cap and the linear support body, where the plurality of support members structurally configured to support the distal cap at the aneurism.

In another example, the linear support body has a lower porosity than the transverse flow section and a higher porosity than the distal cap.

In another example, the distal cap has a porosity that allows sufficient blood flow through into the aneurysm to inhibit thrombosis from forming on the distal cap and that restricts sufficient blood flow into the aneurism to facilitate endothelization on the distal cap.

In another example, the distal cap has a porosity of from about 15% to about 55%.

In another example, the distal cap has a porosity of from about 30% to about 40%.

In another example, the distal cap, the transverse flow section, and the linear support body are comprised of braided wires.

In another example, the proximal transverse opening is an opening in a braiding pattern of the braided wires.

In another example, the delivery lumen is sized to maintain the flow diverter in the undeployed configuration.

In another example, the distal-facing region of the proximal transverse opening is held mechanically engaged with the proximal transverse edge by the delivery lumen.

In another example, the proximal transverse edge of the flow diverter anchor is structurally configured to mechanically disengage from the proximal transverse opening when the delivery lumen is withdrawn to fully expose a proximal end of the linear support body.

In another example, wherein the proximal transverse edge of the flow diverter anchor is structurally configured to mechanically disengage from the proximal transverse opening when the delivery lumen is withdrawn to expose a proximal end of the flow diverter sufficiently to allow the proximal transverse opening to lift off of the proximal transverse edge between the flow diverter anchor and the delivery lumen.

In another example, the proximal transverse edge of the flow diverter anchor and the delivery lumen are structurally configured to maintain the capacity to withdraw the flow diverter into the delivery lumen when the flow diverter is at least 70% deployed.

In another example, the proximal transverse edge of the flow diverter anchor and the delivery lumen are structurally configured to maintain the capacity to withdraw the flow diverter into the delivery lumen when the flow diverter is at least 80% deployed.

In another example, the proximal transverse edge of the flow diverter anchor and the delivery lumen are structurally configured to maintain the capacity to withdraw the flow diverter into the delivery lumen when the flow diverter is at least 90% deployed.

In another example, the braided wires are braided wire bundles.

In another example, the flow diverter delivery system further includes a plurality of wire bundle slack adjusters coupled between the proximal end of the distal cap and a distal end of the transverse flow section, wherein each of the plurality of wire bundle slack adjusters is structurally configured to provide sufficient slack to allow each associated wire bundle to stretch out along a central axis of the flow diverter in the undeployed configuration and to then take up sufficient slack to allow the flow diverter to deploy into its original shape in the deployed configuration.

In another example, each of the plurality of wire bundle slack adjusters transitions at its proximal end to a primary wire bundle of a plurality of wire bundles to form the transverse flow section.

What is claimed is:

1. A flow diverter delivery system, comprising:
a catheter sized for insertion and movement through a blood vessel, the catheter having a delivery lumen;
a pusher wire slidably disposed within the delivery lumen;
a distal guide linearly coupled to a distal end of the pusher wire;
a flow diverter having an undeployed configuration and a deployed configuration, the flow diverter, in the deployed configuration, including;
a low-porosity distal cap having an outer convex shape structurally configured to be longitudinally positionable adjacent a luminal wall of a blood vessel bifurcation at an aneurysm;
a transverse flow section coupled to a proximal end of the distal cap; and
a linear support body coupled to a proximal end of the transverse flow section and having a proximal transverse opening with a distal facing region,
wherein the low-porosity distal cap is structurally configured to divert at least a portion of blood received from the linear support body through the transverse flow section;
a flow diverter anchor coupled to the distal guide and having a proximal transverse edge, the proximal transverse edge mechanically engaged to the distal-facing region of the proximal transverse opening of the flow diverter in the undeployed configuration, wherein the proximal transverse edge is further structurally configured such that radial movement of the proximal transverse opening away from the transverse edge disengages the flow diverter from the flow diverter anchor.

2. The flow diverter delivery system of claim 1, wherein the transverse flow section is comprised of a plurality of support members coupled between the distal cap and the linear support body, the plurality of support members structurally configured to support the distal cap at the aneurism.

3. The flow diverter delivery system of claim 1, wherein the linear support body has a lower porosity than the transverse flow section and a higher porosity than the distal cap.

4. The flow diverter delivery system of claim 1, wherein the distal cap has a porosity that allows sufficient blood flow through into the aneurysm to inhibit thrombosis from forming on the distal cap and that restricts sufficient blood flow into the aneurism to facilitate endothelization on the distal cap.

5. The flow diverter delivery system of claim 1, wherein the distal cap has a porosity of from about 15% to about 55%.

6. The flow diverter delivery system of claim 1, wherein the distal cap has a porosity of from about 30% to about 40%.

7. The flow diverter delivery system of claim 1, wherein the distal cap, the transverse flow section, and the linear support body are comprised of braided wires.

8. The flow diverter delivery system of claim 7, wherein the proximal transverse opening is an opening in a braiding pattern of the braided wires.

9. The flow diverter delivery system of claim 1, wherein the delivery lumen is sized to maintain the flow diverter in the undeployed configuration.

10. The flow diverter delivery system of claim 9, wherein the distal-facing region of the proximal transverse opening is held mechanically engaged with the proximal transverse edge by the delivery lumen.

11. The flow diverter delivery system of claim 10, wherein the proximal transverse edge of the flow diverter anchor is structurally configured to mechanically disengage from the proximal transverse opening when the delivery lumen is withdrawn to fully expose a proximal end of the linear support body.

12. The flow diverter delivery system of claim 10, wherein the proximal transverse edge of the flow diverter anchor is structurally configured to mechanically disengage from the proximal transverse opening when the delivery lumen is withdrawn to expose a proximal end of the flow diverter sufficiently to allow the proximal transverse opening to lift off of the proximal transverse edge between the flow diverter anchor and the delivery lumen.

13. The flow diverter delivery system of claim 10, wherein the proximal transverse edge of the flow diverter anchor and the delivery lumen are structurally configured to maintain the capacity to withdraw the flow diverter into the delivery lumen when the flow diverter is at least 70% deployed.

14. The flow diverter delivery system of claim 10, wherein the proximal transverse edge of the flow diverter anchor and the delivery lumen are structurally configured to maintain the capacity to withdraw the flow diverter into the delivery lumen when the flow diverter is at least 80% deployed.

15. The flow diverter delivery system of claim 10, wherein the proximal transverse edge of the flow diverter anchor and the delivery lumen are structurally configured to maintain the capacity to withdraw the flow diverter into the delivery lumen when the flow diverter is at least 90% deployed.

16. The flow diverter delivery system of claim 7, wherein the braided wires are braided wire bundles.

17. The flow diverter delivery system of claim 16, further comprising a plurality of wire bundle slack adjusters coupled between the proximal end of the distal cap and a distal end of the transverse flow section, wherein each of the plurality of wire bundle slack adjusters is structurally configured to provide sufficient slack to allow each associated wire bundle to stretch out along a central axis of the flow diverter in the undeployed configuration and to then take up sufficient slack to allow the flow diverter to deploy into its original shape in the deployed configuration.

18. The flow diverter delivery system of claim 17, wherein each of the plurality of wire bundle slack adjusters transitions at its proximal end to a primary wire bundle of a plurality of wire bundles to form the transverse flow section.
